(19) United States
(12) Patent Application Publication
(10) Pub. No.: US 2009/0011435 A1
(43) Pub. Date: Jan. 8, 2009

(54) ASSAYS FOR DETECTING ANTIBODIES TO THERAPEUTICS

(76) Inventor: Thomas L. Cantor, El Cajon, CA (US)

Correspondence Address: MORRISON & FOERSTER LLP, 12531 HIGH BLUFF DRIVE, SUITE 100, SAN DIEGO, CA, 92130-2040, US

(21) Appl. No.: 12/136,268

(22) Filed: Jun. 10, 2008

Related U.S. Application Data
(60) Provisional application No. 60/947,867, filed on Jul. 3, 2007.

(51) Int. Cl. G01N 33/53; G01N 33/566

(52) U.S. Cl. 435/7.9; 436/501

(57) ABSTRACT

The disclosed invention relates to methods and devices for detecting a biologic in a test sample. In particular, the disclosed assay utilizes EPO as a target to detect endogenous anti-EPO antibodies in a sample and does not manipulate or modify the target EPO antibody.

### CROSS-REFERENCE TO RELATED APPLICATIONS

This application claims priority from U.S. provisional application 60/947,867, filed Jul. 3, 2007. The contents of this document are incorporated herein by reference.

### TECHNICAL FIELD

The disclosed invention relates to methods of detecting antibodies generated against a therapeutic biological composition, such as a recombinant protein, a hormone, or an antibody.

### BACKGROUND ART

The ability to produce proteins recombinantly has provided a number of new treatments for age-old aliments. Take anemia, for example. Anemia is a lack of red blood cells in the bloodstream that results in diminished oxygen-carrying capacity. It is a common complication of end-stage renal disease, cancer and other disease conditions. The introduction of recombinant human erythropoietin into clinical practice has been an important development in the treatment of anemia. The Medicare program has consistently increased the amount of money spent annually on erythropoietin therapy in conjunction with renal disease since 1991 and the trend does not appear to be tapering. See FIG. 1. However, poor responsiveness to erythropoietin therapy is commonly known to complicate the treatment of anemia in many patients. In some patients, anemia can be unresponsive to erythropoietin therapy, even after repeated dose escalations. Zhang et al., Am. J. Kid. Dis. 44:866-876 (2004). Unresponsiveness not only leaves the patient untreated, but raises the cost of treatment and leaves the patient at risk of developing an auto immunity to endogenous erythropoietin as well as complications arising from insoluble immune complexes in the blood.

Resistance or hypo-responsiveness to erythropoietin therapy has been associated with an increase in mortality, even when mortality is adjusted for hematocrit. See FIG. 2. In order to reduce mortality, it may be crucial to aggressively investigate and correct the cause of resistance or hypo-responsiveness to erythropoietin therapy. In many cases, the underlying cause of erythropoietin resistance or hypo-responsiveness cannot be ameliorated simply by escalating the dose of erythropoietin. Proposed causes of such resistance include antibodies to erythropoietin as well as iron deficiency, inadequate dialysis, and malnutrition-inflammation complex syndrome. Zhang et al., Am. J. Kid. Dis. 44:866-876 (2004), see also FIGS. 3 and 4.

Recently, it has been recognized that a form of anemia, sometimes called antibody-mediated pure red cell aplasia (PRCA), may develop when a patient's own immune system mounts a neutralizing antibody response to therapeutic erythropoietin. These neutralizing antibodies may even cross-neutralize endogenous erythropoietin, leading to a serious state of overwhelming erythropoietin resistance and transfusion dependence. The diagnosis of antibody-mediated PRCA would depend, in part, on the availability of a sensitive and specific method for detecting serum anti-erythropoietin antibodies. Thorpe and Swanson, Nephrol. Dial. Transplant. 20 (Suppl 4):iv16-iv22 (2005).

A variety of assays have been used to detect anti-erythropoietin antibodies. In the past, these have included inhibition of heme synthesis in normal bone marrow, immunocytofluorescence, and cytotoxic release of ⁵⁹Fe from labeled erythroblasts. Current assays include enzyme-linked immunosorbent assays (ELISAs), radioimmunoprecipitation assays (RIPs), surface plasmon resonance (SPR), and various bioassays. Each of these assays possess characteristic benefits and limitations in terms of specificity, sensitivity, and ease of use. Most of the current assays require some manipulation of the erythropoietin used to detect anti-erythropoietin antibodies. To date, a universal standardized assay has not been established. Thorpe and Swanson, Nephrol. Dial. Transplant. 20 (Suppl 4):iv16-iv22 (2005).

Indirect and bridging ELISA techniques have been used to detect anti-erythropoietin antibodies. In the indirect ELISA technique, serial dilutions of a patient's plasma are incubated in wells of microtitre plates that are coated with erythropoietin. Any anti-erythropoietin antibodies present in the plasma then bind to the erythropoietin that is immobilized on the plate wall. A secondary, labeled antibody is added to detect any anti-erythropoietin antibodies bound to the immobilized erythropoietin. The bridging ELISA technique requires even more manipulation of erythropoietin. As in the more conventional indirect technique, serial dilutions of a patient's plasma are incubated in wells of microtitre plates that are coated with erythropoietin. Anti-erythropoietin antibodies present in the plasma bind to the erythropoietin that is immobilized on the plate wall. However, the bridging technique then requires the addition of labeled erythropoietin, which binds to the second, open antigen binding site of the anti-erythropoietin antibody. Immobilization of the erythropoietin on the plate wall may alter the conformation of the protein. Furthermore, in the bridging technique, labeling of the erythropoietin may alter or denature the protein. Therefore, manipulation of the erythropoietin used in ELISA techniques may affect the specificity and sensitivity of the assay. Thorpe and Swanson, Nephrol. Dial. Transplant. 20 (Suppl 4):iv16-iv22 (2005).

In the RIP assay, serum anti-erythropoietin antibodies are allowed to bind to ¹²⁵I-labeled erythropoietin in solution. The resulting complexes are captured by a solid phase anti-globulin reagent (e.g., protein A-Sepharose beads) and pelleted by centrifugation for analysis. The amount of radioactivity in the pelleted samples corresponds positively with the relative concentration of erythropoietin-specific antibodies in the serum samples. However, the radiolabeling of the erythropoietin may alter or denature the protein, and may therefore affect the accuracy of the assay. Thorpe and Swanson, Nephrol. Dial. Transplant. 20 (Suppl 4):iv16-iv22 (2005).

SPR, sometimes called the BIAcore assay, relies on the change in the angle of incident light reflected off the gold surface of a biosensor chip as a function of the amount of protein mass accumulating on the sensor chip surface. Erythropoietin is bound to the sensor chip surface and any anti-erythropoietin antibodies present in the serum sample will bind to the immobilized erythropoietin. The resulting protein mass alters the angle of reflection of light reaching the photometric detectors. As in the ELISA technique, immobilization of erythropoietin may alter the conformation of the protein, affecting the accuracy of the assay. Thorpe and Swanson, Nephrol. Dial. Transplant. 20 (Suppl 4):iv16-iv22 (2005).

Bioassays typically utilize bone-marrow derived erythroid cells or certain cell lines that proliferate in the presence of erythropoietin. This erythropoietin-dependent cellular proliferation may be blocked by neutralizing anti-erythropoietin antibodies present in a patient's serum. Although bioassays do not necessarily require manipulation of the erythropoietin used, they present a number of formidable disadvantages compared to the other techniques discussed above. For example, bone marrow samples may be available in limited quantities. Different cell lines have different sensitivities to erythropoietin and the neutralizing effects of anti-erythropoietin antibodies, resulting in variable results even with the same serum sample if different cell lines are utilized. Finally, these bioassays often require long incubation periods, sometimes up to 14 days. Thorpe and Swanson, Nephrol. Dial. Transplant. 20 (Suppl 4):iv16-iv22 (2005).

The slate of currently available assays clearly illustrates the need for an assay that requires no manipulation of the erythropoietin used, no need for harvesting bone marrow cells, and no prolonged incubation period.

### DISCLOSURE OF THE INVENTION

The disclosed invention relates to a method for detecting an endogenous antibody to therapeutic compounds, comprising the steps of providing a test sample, a capture agent, a bait sample which is the unmanipulated therapeutic compound and mixing them to prepare an initial mixture. In a preferred embodiment, a secondary mixture is prepared by adding a signaling agent that binds to endogenous antibodies bound to the bait in the initial mixture. A signal from the signaling agent is detected in the secondary mixture. In an alternative embodiment, the signaling agent is included in the initial mixture. Preferred examples of a therapeutic compound include erythropoietin (EPO), growth hormone and insulin.

In an preferred embodiment, the capture agent is a protein or peptide capable of specifically binding to the bait. In one embodiment of the invention the capture agent is an antibody which binds the bait. The capture agent antibody can be a polyclonal antibody or a monoclonal antibody. The capture agent antibody can also be an antibody fragment which binds to the bait. Examples of antibody fragments include an Fab, a F(ab′)₂, a Fv or a Sfv fragment. In an alternative embodiment, the capture agent is a receptor or ligand binding fragment thereof which is capable of bind the bait with specificity.

[In another embodiment of the invention the signaling agent is a protein or peptide capable of specifically binding to the bait in a manner which does not inhibit the capture agent from binding to the bait. In a preferred embodiment, the signaling agent is an antibody which binds the bait. Preferably the signaling agent antibody binds to an epitope other than that to which the capture agent antibody binds. The signaling agent antibody can be a polyclonal antibody or a monoclonal antibody. The signaling agent antibody can also be an antibody fragment which binds to the bait. Examples of antibody fragments include an Fab, a F(ab′)₂, a Fv or a Sfv fragment. In another embodiment, the signaling agent can comprise multiple components such as a primary antibody and a secondary antibody. For example, a primary antibody which binds to the bait and is labeled with a marker against which a secondary antibody binds, wherein the secondary antibody is labeled with a signal generating agent is also contemplated for use with the present invention. An avidin/biotin system is an example of a signaling agent comprising multiple components.

The signaling agent is preferably labeled with a signal-generating compound. Examples of a signal-generating compound include chromogens, radioisotopes, chemiluminescent compounds, and enzymes. In one embodiment, the enzyme is horseradish peroxidase.

### BRIEF DESCRIPTION OF THE DRAWINGS

FIG. 1 shows a bar graph of Medicare Epo costs from 1991-2003.

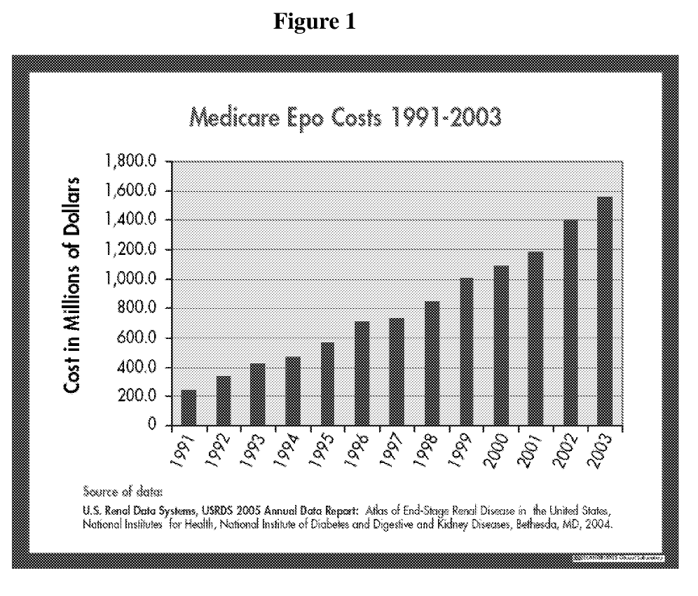

FIG. 2 shows a bar graph of mortality versus hematocrit versus Epo dose.

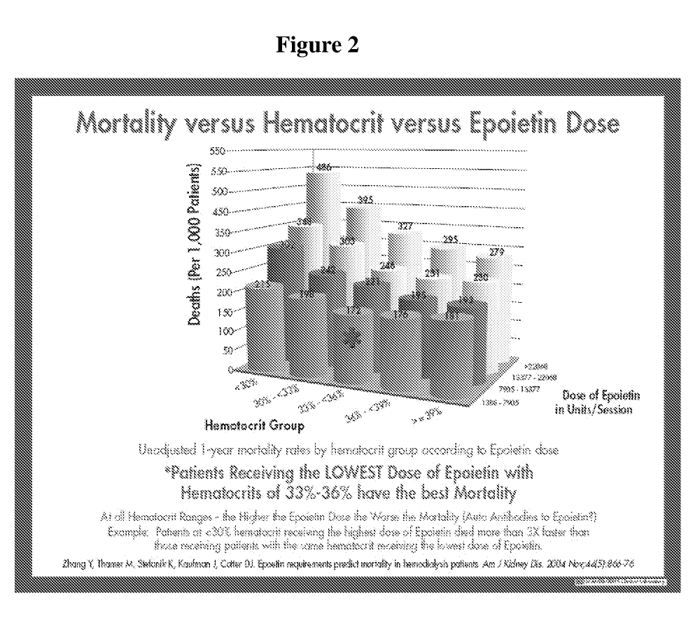

FIG. 3 shows a survey in bar graph form of antibodies to Epo in 48 ESRD patients.

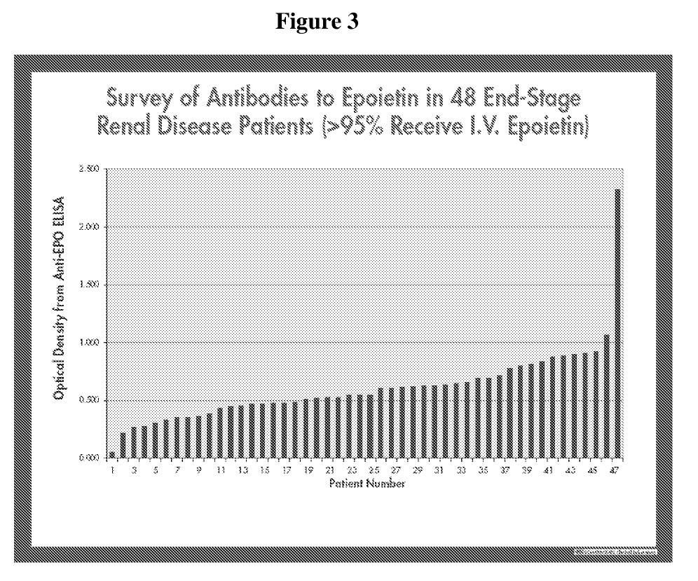

FIG. 4 shows a table of anti-Epo activity in ESRD patient plasma.

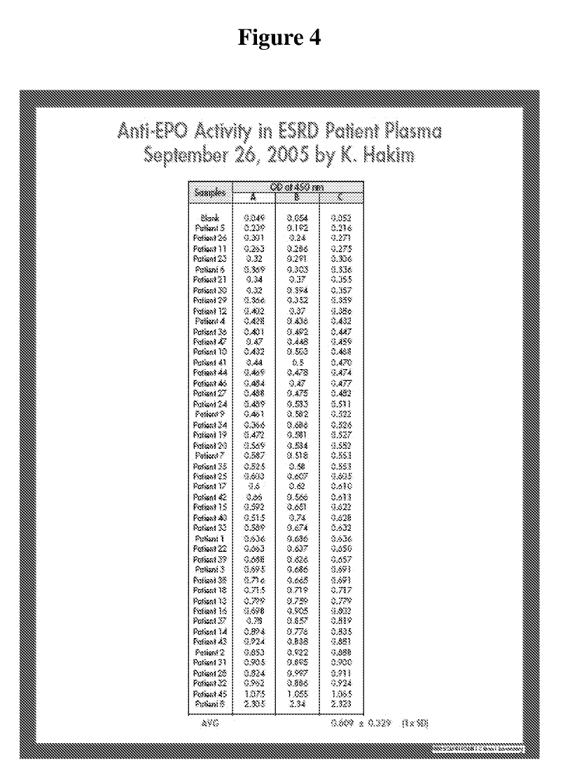

FIG. 5 shows a schematic view of an embodiment of the disclosed assay

### MODES OF CARRYING OUT THE INVENTION

The disclosed invention relates to an immunoassay for detecting antibodies raised by a host subject receiving a therapeutic biologic compound. A biologic compound or biologic is a preparation such as a recombinant or purified protein, an antigen, or an antibody or fragment thereof, which has been synthesized, for example from a living organism or their products and used as a diagnostic, preventive, or therapeutic agent. Frequently, a host receiving one or more of these products will generate an immune response against the biologic. One example of this situation is the increase in anti-erythropoietin (EPO) antibodies that occur in individuals who receive this protein. The recombinant product is substantially similar to the naturally occurring form of the protein, which is produced primarily by kidney cells and plays a role in hematopoiesis. As discussed above, it had been recognized that a form of anemia, sometimes called antibody-mediated pure red cell aplasia (PRCA), may develop when a patient's own immune system mounts a neutralizing antibody response to therapeutic EPO. Another example is the development of antibodies against an inhaled form of insulin (EXUBRA). See, e.g., Heise, et al., Diabetes Care. (2005) 28(9):2161-9. However, antibodies developed against the inhaled form of insulin did not appear to retard the efficacy of the inhaled biologics. Thus, the ability to monitor levels of endogenous antibodies against therapeutic biologics represents a useful diagnostic and therapeutic tool. The disclosed method can also be used to detect the illicit use of biologics.

The disclosed invention seeks to exploit a frequently overlooked aspect of immunoassays. Often, antigens used in immunoassays are directly manipulated to facilitate detection of antibodies made thereto. For example, to determine if anti-EPO antibodies have been generated in a person suspected of taking EPO, one might dry or otherwise attach EPO to the surface of an assay plate for use as bait to lure antibodies in a sample taken from that person for detection. While this approach is simple it suffers from significant limitations. One limitation is that antigens directly bound to the surface of an assay plate are likely to undergo a change in conformation. This change or distortion of the antigen may mask or completely destroy epitopes on the surface of the target molecule. By masking these epitopes, immunoassays looking to detect antibodies against the antigen of interest may reflect false negative readings caused by the distortion of the antigen. The disclosed invention seeks to overcome this limitation by using an indirect method of detecting antibodies to a particular biologic target. In a preferred embodiment, the assay uses the so-called sandwich assay approach for the detection of an endogenous antibody against a biologic.

Exemplary Antigens

Many biologics are in use today and detection of the presence of these biologics presents a challenge to standard methods, such as urinalysis. Examples of antigens of interest include erythropoietin (AMGEN), alpha-1-protease inhibitor (BAXTER HEALTHCARE CORP), anti-hemophilic factor (BAYER CORP), human growth factor, bovine growth factor, coagulation factor VIIa, and coagulation factor IX.

Anti-Biologic Antibody Detection Immunoassay

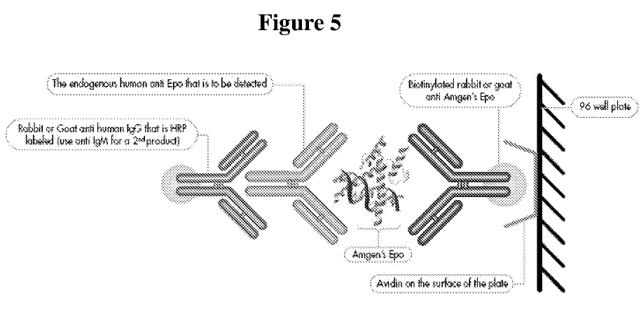

A schematic of a sandwich assay is shown in FIG. 5. Elements employed in such an assay include a capture antibody attached, directly or indirectly to a solid support, an endogenous antibody which is generated against the biologic of interest, a signal antibody having a label attached thereto, and an exogenous source of the biologic. In preferred embodiments, one or more capture antibodies which are known to bind selectively to different epitopes on the biologic to maximize detection of endogenous antibodies.

A preferred embodiment of the disclosed invention is used to detect the presence of anti-EPO antibodies with an exogenous source of erythropoietin (EPO). In certain embodiments capture antibodies are selected that bind to different epitopes on EPO. Using different epitope detecting antibodies permits the detection of endogenous antibodies that happen to bind to the same or similar epitopes recognized by endogenous antibodies.

In one embodiment of the disclosed invention, capture antibodies that specifically bind EPO, or portions thereof, are coated on a solid phase. Bait in the form of EPO is incubated with the solid phase. The test or biological sample is then contacted with the solid phase. In an alternative embodiment, the bait EPO can be incubated with the test or biological sample prior to incubation with the solid phase displaying the capture antibody. If antibodies are present in the sample, such antibodies bind to the bait on the solid phase and are then detected. In a preferred embodiment, a signal antibody which binds to human antibodies in the sample and is labeled or conjugated to a signal-generating compound or label is added to the bound antibody. Should the signal antibody bind to a bound endogenous anti-EPO antibody present in the test sample, the signal-generating compound generates a measurable signal. Such signal then indicates presence of the endogenous anti-EPO antibody in the test sample.

Examples of solid phases used in diagnostic immunoassays are porous and non-porous materials, latex particles, magnetic particles, microparticles (see U.S. Pat. No. 5,705,330, which is hereby incorporated by reference in its entirety), beads, membranes, microtiter wells and plastic tubes. The choice of solid phase material and method of labeling the antigen or antibody present in the conjugate, if desired, is determined based upon desired assay format performance characteristics.

As noted above, the signal antibody comprises an antibody or binding fragment, attached to a signal-generating compound or label. This signal-generating compound or “label” is in itself detectable or may be reacted with one or more additional compounds to generate a detectable product. Examples of signal-generating compounds include chromogens, radioisotopes (e.g., ¹²⁵I, ¹³¹I, ³²P, ³H, ³⁵S and ¹⁴C), chemiluminescent compounds (e.g., acridinium), particles (visible or fluorescent), nucleic acids, complexing agents, or catalysts such as enzymes (e.g., alkaline phosphatase, acid phosphatase, horseradish peroxidase, beta-galactosidase and ribonuclease). In the case of enzyme use (e.g., alkaline phosphatase or horseradish peroxidase), addition of a chromo-, fluro-, or lumo-genic substrate results in generation of a detectable signal. Other detection systems such as time-resolved fluorescence, internal-reflection fluorescence, amplification (e.g., polymerase chain reaction) and Raman spectroscopy are also useful.

Examples of biological fluids which may be tested by the above immunoassays include plasma, serum, cerebrospinal fluid, saliva, tears, nasal washes or aqueous extracts of tissues and cells.

The antibodies which are coated on the solid phase as well as the signal generating antibodies may be, as noted above, monoclonal antibodies or polyclonal antibodies. For example, if one chooses to utilize monoclonal antibodies, commercial forms thereof are available, for example, anti-EPO-16 and anti-EPO-26, which detect non-overlapping epitopes on the EPO molecule with high affinity. (STEMCELL TECHNOLOGIES). (For a discussion of the manner in which monoclonal antibodies may be created, see Kohler and Milstein, Nature (1975) 256:494, and reviewed in Monoclonal Hybridoma Antibodies: Techniques and Applications, ed. Hurrell (CRC Press, Inc., 1982); see also J. W. Goding in Monoclonal Antibodies: Principles and Practice (Academic Press, N.Y., 1983; see also U.S. Pat. No. 5,753,430).

Additionally, it should also be noted that the initial capture antibody (for detecting EPO) used in the immunoassay may be covalently or non-covalently (e.g., ionic, hydrophobic, etc.) attached to the solid phase. Linking agents for covalent attachment are known in the art.

Other assay formats which may be used for purposes of the disclosed invention, in order to detect anti-biologic antibodies without modifying the biologic itself, include, for example, the use of paramagnetic particles in, for example, an ARCHITECT assay (Frank Quinn, The Immunoassay Handbook, Second edition, edited by David Wild, pages 363-367, 2001). Such formats are known to those of ordinary skill in the art.

It should also be noted that the elements of the assay described above are particularly suitable for use in the form of a kit. The kit may also comprise one container such as vial, bottles or strip, with each container with a pre-set solid phase, and other containers containing the respective conjugates. These kits may also contain vials or containers of other reagents needed for performing the assay, such as washing, processing and indicator reagents.

The ordinarily skilled artisan can appreciate that the present invention can incorporate any number of the preferred features described above. All publications or unpublished patent applications mentioned herein are hereby incorporated by reference thereto. Other embodiments of the present invention are not presented here which are obvious to those of ordinary skill in the art, now or during the term of any patent issuing from this patent specification, and thus, are within the spirit and scope of the present invention.

The following examples are offered to illustrate but not to limit the invention.

### EXAMPLE 1

### Detection of Human Anti-Erythropoietin Antibodies

One hundred (100) microliters of serum from a patient with anti-erythropoietin antibodies is added to a tube. Ten (10) nanograms of EPO (AMGEN) or PROCRIT (J&J) is also added to the tube. The mixture is incubated overnight at room temperature with mild agitation. Next one microgram of biotinylated anti-EPO antibody is added to the mixture, which is again incubated overnight at room temperature with mild agitation. The mixture is then transferred from the tube to an avidin coated well in a 96 well plate. One (1) microgram of an antibody which recognizes human antibodies is added to the mixture. This antibody is the signal antibody, which is labeled with horse radish peroxidase (HRP). The plate is incubated for two (2) hours at room temperature with mild agitation. The wells of the plate are then washed and the HRP substrate is added. The presence or absence of a signal from the signal antibody is detected.

1. A method for detecting an endogenous antibody to a biologic, comprising:
providing to a test sample a capture agent and a bait sample of the biologic to prepare an initial mixture;
preparing a secondary mixture by adding a signaling agent that binds to an endogenous antibody that binds to the bait in the initial mixture; and
detecting signal from the signaling agent in the secondary mixture.

2. The method of claim 1, wherein the capture agent is an antibody which binds the bait.

3. The method of claim 2, wherein the antibody is a polyclonal antibody or a monoclonal antibody.

4. The method of claim 2, wherein the antibody is an antibody fragment selected from the group consisting of wherein the fragment is an Fab, a F(ab′)₂, a Fv and a Sfv fragment.

5. The method of claim 1, wherein the signaling agent is an antibody which binds the endogenous antibody which binds the bait.

6. The method of claim 5, wherein the antibody is a polyclonal antibody or a monoclonal antibody.

7. The method of claim 5, wherein the antibody is an antibody fragment selected from the group consisting of wherein the fragment is an Fab, a F(ab′)₂, a Fv and a Sfv fragment.

8. The method of claim 5, wherein the antibody is an antibody is labeled with a signal-generating compound.

9. The method of claim 8, wherein the compound is selected from the group consisting of a chromogen, a radioisotope, a chemiluminescent compound, and an enzyme.

10. The method of claim 9, wherein the enzyme is horseradish peroxidase.

11. The method of claim 1, wherein the biologic is selected from the group consisting of erythropoietin (EPO), insulin, alpha-1-proteinase inhibitor, human growth hormone, and bovine growth hormone.
